(19) United States
(12) Patent Application Publication
(10) Pub. No.: US 2026/0334963 A1
(43) Pub. Date: Oct. 29, 2026

(54) THROMBUS REMOVAL SYSTEM CONSOLES AND ASSOCIATED USER INTERFACES

(71) Applicant: Shifamed Holdings, LLC, Campbell, CA (US)

(72) Inventors: Aadel AL-JADDA, San Carlos, CA (US); Praveen Krishna DALA, San Ramon, CA (US)

(21) Appl. No.: 19/483,840

(22) PCT Filed: May 13, 2024
(86) PCT No.: PCT/US2024/029163

§ 371 Date: Nov. 12, 2025

Related U.S. Application Data
(60) Provisional application No. 63/502,042, filed on May 12, 2023.

(51) Int. Cl. A61B 17/22; A61B 17/00; A61M 1/00; A61M 39/28

(52) U.S. Cl. CPC A61B 17/22; A61B 2017/00199; A61B 2017/22079; A61M 1/77; A61M 39/28; A61M 2205/0244; A61M 2205/3327; A61M 2205/3331

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus.

### PRIORITY CLAIM

This patent application claims priority to U.S. provisional patent application No. 63/502,042, titled “THROMBUS REMOVAL SYSTEM CONSOLES AND ASSOCIATED USER INTERFACES,” and filed on May 12, 2023, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the tortuous vascular anatomy, may not be effective in removing thrombotic material, and/or may lack the ability to provide sensor data or other feedback to the clinician during the thrombectomy procedure. Existing thrombectomy devices operate based on simple aspiration which works sufficiently for certain clots but is largely ineffective for difficult, organized clots. Many patients presenting with deep vein thrombus (DVT) are left untreated as long as the risk of limb ischemia is low. In more urgent cases, they are treated with catheter-directed thrombolysis or lytic therapy to break up a clot over the course of many hours or days. More recently other tools like clot retrievers have been developed to treat DVT and pulmonary embolism (PE), but these tools are not being widely adopted because of their limited effectiveness and additional costs versus aspiration or the standard of case. Other recent developments focus on slicing or macerating the clot, but these mechanisms are designed to reduce the risk of the catheter clogging and do not address the problem of tough, large, organized clots. There remains the need for a device to address these and other problems with existing venous thrombectomy including, but not limited to, a fast, easy-to-use, and effective device for removing a variety of clot morphologies.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports and fluid streams of a thrombus removal system according to embodiments of the present technology.

FIGS. 4A-4C illustrate various embodiments of a thrombus removal system including a saline source, an aspiration system, and one or more controls for controlling irrigation and/or aspiration of the system.

FIGS. 5A-5B show embodiments of a console of a thrombectomy system.

FIGS. 6A-6D illustrate views of a vacuum cassette configured to interface with the console and integrate a pressure sensor and pinch valve into the system.

FIGS. 7A-7E illustrate views of an irrigation pump configured to deliver irrigation fluid and/or jetting fluid into a thrombectomy catheter from the console.

FIGS. 8A-8G illustrate views of a user interface of a thrombectomy console. The views can be displayed on a screen or display of the console, for example.

### SUMMARY OF THE DISCLOSURE

A thrombus removal is provided, comprising an elongate shaft comprising a working end, at least one fluid lumen in the elongate shaft, and two or more apertures disposed at or near the working end, the two or more apertures in fluid communication with the least one fluid lumen and configured to generate two or more fluid streams to mechanically fractionate a target thrombus.

In one aspect, a thrombus removal system is provided, comprising a thrombectomy catheter having an elongate shaft, an aspiration lumen extending along the elongate shaft, and at least one fluid lumen extending along the elongate shaft; a console configured to be coupled to the thrombectomy catheter, the console including: a vacuum source adapted to be fluidly coupled to the aspiration lumen of the thrombectomy catheter with a vacuum line; an irrigation source and pump configured to provide a flow of fluid to the at least one fluid lumen of the thrombectomy catheter with a fluid line; and a pinch valve assembly configured to receive the vacuum line, wherein the pinch valve assembly is configured to selectively close off the aspiration lumen from the vacuum source.

In some aspects, the pinch valve assembly comprises a pinch valve disposed on the console.

In one aspect, the pinch valve assembly is configured receive a vacuum line cassette that includes a pinch valve portion of the vacuum line configured to interface with the pinch valve on the console.

In some aspects, the vacuum line cassette includes a pressure transducer configured to measure a pressure in the aspiration lumen of the thrombectomy catheter.

In other aspects, the pressure transducer comprises a MEMS pressure transducer.

In some aspects, the system includes a pressure transducer wire couped to the pressure transducer and extending out of the vacuum line cassette.

In some aspects, the console includes a pressure wire connector configured to receive the pressure transducer wire to operatively couple the pressure transducer to the console.

In one aspect, the vacuum line cassette is disposable.

In some aspects, the irrigation pump is at least partially disposed in the console.

In other aspects, the irrigation pump comprises a disposable component and a permanent component.

In some aspects, the system includes a display configured to guide a user through a thrombectomy procedure.

In one aspect, the display is configured to present an indicator showing a status of thrombectomy catheter components that are connected to the console.

In some aspects, the display is configured to present an indicator showing a clot engagement status of the thrombectomy catheter.

A thrombectomy system console is provided, comprising: a vacuum source; a pinch valve assembly comprising a pinch valve and a mounting bracket, the pinch valve assembly being configured to receive a vacuum cassette having an inlet fluidly coupled to a thrombectomy device and an outlet fluidly coupled to the vacuum source, the pinch valve being operatively coupled to a portion of tubing in the vacuum cassette when the vacuum cassette is inserted into the mounting bracket; and one or more processors operatively coupled to the pinch valve assembly and configured to selectively couple the vacuum source to the thrombectomy device.

In some aspects, the vacuum cassette is fluidly coupled to an aspiration lumen of a thrombectomy device.

In one aspect, the pinch valve comprises a pump head.

In one aspect, the mounting bracket comprises one or more alignment features configured to interface with one or more corresponding surfaces of the vacuum cassette.

In some aspects, the vacuum cassette includes an inlet and an outlet.

In other aspects, the inlet is coupled to a vacuum line that extends to an aspiration lumen of the thrombectomy device.

In some aspects, the outlet is coupled to a vacuum line that extends to the vacuum source.

In additional aspects, the pump head fits within an opening of the vacuum cassette.

In some aspects, a pressure transducer is disposed in the vacuum cassette between the inlet and the outlet.

In another aspect, a pressure transducer wire extends from the pressure transducer out of the vacuum cassette.

A vacuum cassette for operatively coupling a vacuum source to an aspiration lumen of a thrombectomy device is provided, comprising: a cassette body; a section of tubing extending through the cassette body between an inlet and an outlet; an opening in the cassette body configured to receive a pinch valve on a console of a thrombectomy system for selectively clamping the section of tubing; and at least one protrusion in the cassette body near the opening and the section of tubing, the at least one protrusion being configured to engage with the section of tubing when compressed by the pinch valve.

In some aspects, the inlet is configured to be coupled to the aspiration lumen of the thrombectomy device with a vacuum line.

In other aspects, the outlet is configured to be coupled to the vacuum source with a vacuum line.

In one aspect, the at least one protrusion comprises three protrusions.

In another aspect, a pressure transducer is disposed in the cassette body between the inlet and the opening.

In some aspects, the cassette includes one or more cassette surfaces configured to engage with one or more alignment features on the console of the thrombectomy system to place the section of tubing in operative contact with the pinch valve.

In some aspects, the cassette includes a tube path within the cassette body that includes a circular cross-section along a first length of the section of tubing and a non-circular cross-section along a second length of the section of tubing.

In another aspect, the non-circular cross-section is configured to conform to the section of tubing when it is compressed by the pinch valve.

In some aspects, the non-circular cross-section is ellipsoidal.

In another aspect, the tube path comprises a first tube path on a first side of the opening and a second tube path on a second side of the opening.

A thrombectomy device display is provided, comprising: a display screen having a clot engagement indicator, the clot engagement indicator being configured to show a first indicator when no clot is engaged with a funnel of a thrombectomy device and a second indicator when clot is engaged with the funnel of the thrombectomy device.

In some aspects, the first indicator comprises a graphic of a representative thrombectomy device and funnel with no clot present.

In other aspects, the second indicator comprises a graphic of a representative thrombectomy device and funnel with one or more clots present in the funnel.

### DETAILED DESCRIPTION

This application is related to disclosure in International Application No. PCT/US2021/020915, filed Mar. 4, 2021 (the '915 application), and International Application No. PCT/US2022/033024, filed Jun. 10, 2022 (the '024 application), the disclosures of which are incorporated by reference herein for all purposes. The '915 and '024 applications describe general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment.

Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to render the structure and/or consistency of the clot such that it is more easily transported through the aspiration system (e.g., fragment the thrombus with pressurized fluid), an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, in addition to or alternatively to high pressure fluid the aspiration pump may provide lower pressure fluid within the aspiration lumen near the proximal portion of the thrombus removal system, urging aspiration of thrombus from the distal portion.

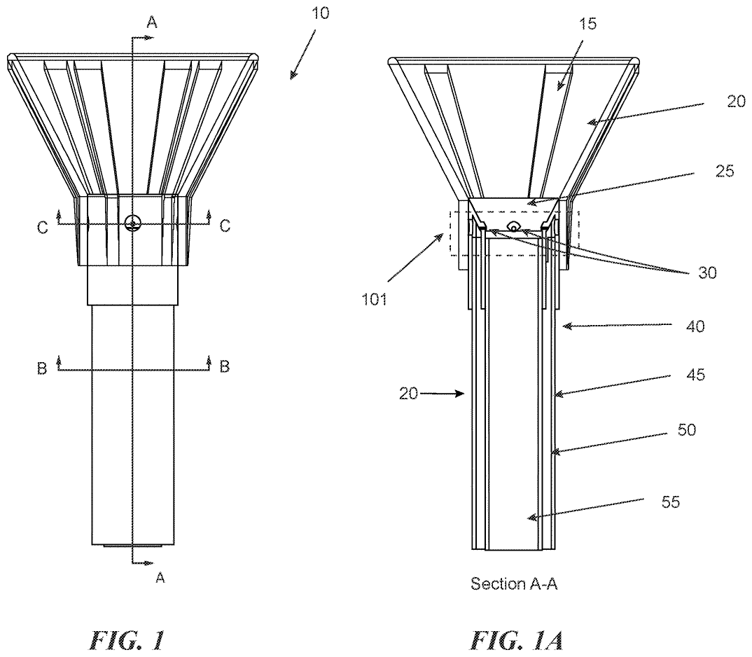

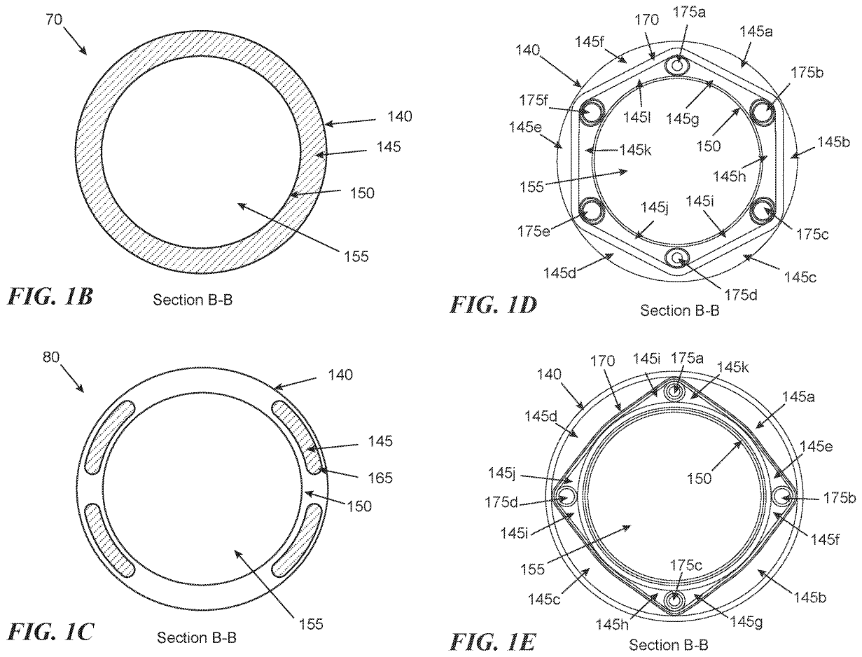

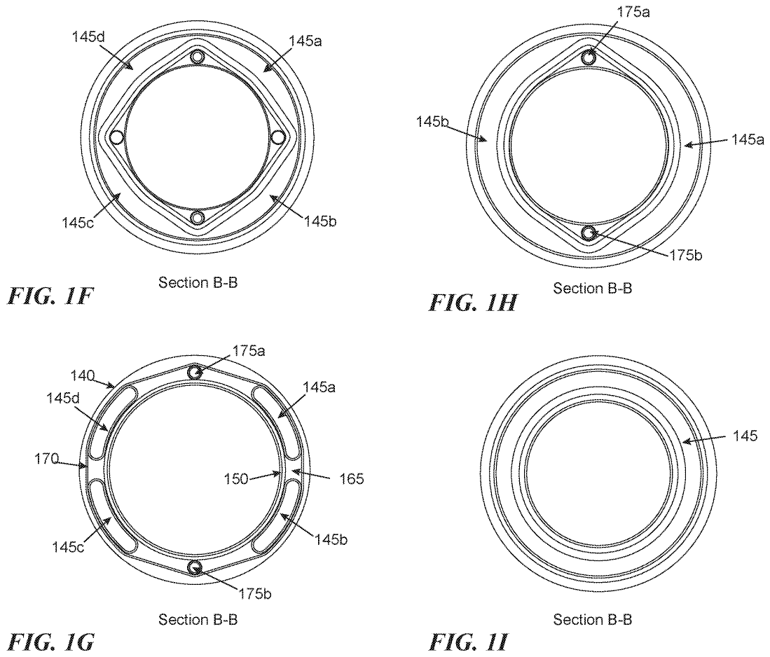

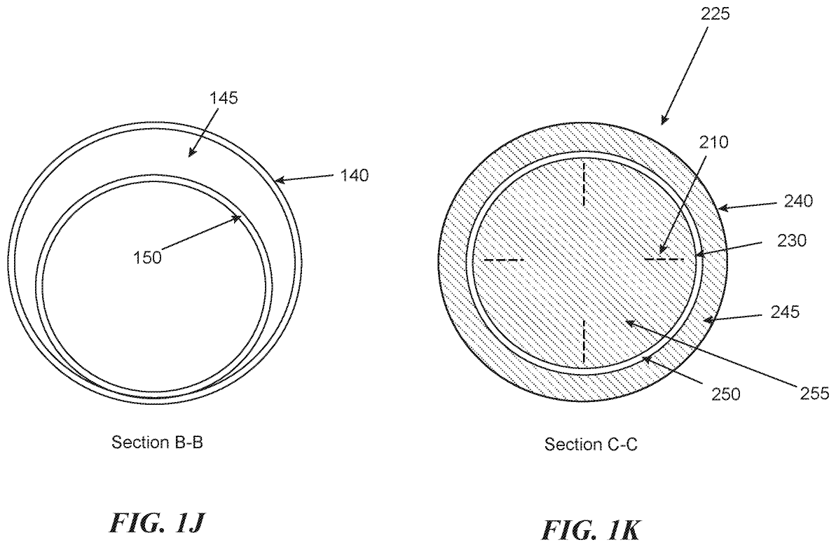

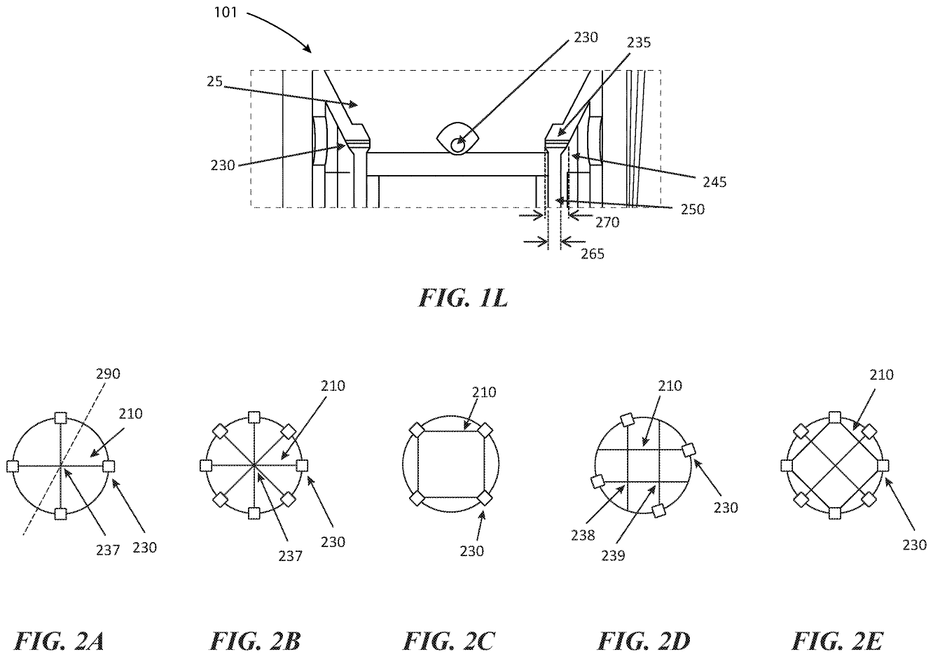

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus within a blood vessel and/or a tissue (e.g., vessel) wall to aid in thrombus fragmentation and/or removal. The funnel can have a variety of shapes and constructions as would be understood by one of skill from the description herein. The thrombus removal system may be delivered through a sheath to a thrombus site in a blood vessel with funnel 20 in a compressed configuration. Funnel 20 may self-expand as it is advanced out of the sheath and/or as the sheath is retracted from the funnel.

The example section A-A in FIG. 1A depicts a double walled thrombus removal device construction having a catheter 22 extending proximally from funnel 20 with an outer wall/tube 40 and an inner wall/tube 50. An aspiration lumen 55 is formed by the inner wall 50 and is centrally located. Aspiration lumen 55 communicates with a vacuum source, as described below. A generally annular volume forms at least one fluid lumen 45 between the outer wall 40 and the inner wall 50. The fluid lumen 45 is adapted for fluid communication with a fluid delivery mechanism, as described below. One or more apertures (e.g., nozzles, orifices, or ports) 30 are positioned in the thrombus removal system to be in fluid communication with the fluid lumen 45 and an irrigation manifold 25 at the base of or within funnel 20. In operation, the ports 30 are adapted to direct (e.g., pressurized) fluid toward thrombus material that is engaged with the distal portion 10 of the thrombus removal system to macerate, fragment, or cut the thrombus material. Aspiration lumen 55 pulls thrombus material along with fluid from ports 30 and blood from the blood vessel proximally to a receptacle outside of the patient, as described below.

In various embodiments, the system can have an average flow velocity within the fluid lumen of up to 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be up to 20 m/s, the peak fluid velocity in the lumen may be up to 30 m/s or more during the pulsing of the fluid source. In some embodiments, the jets or apertures are no smaller than 0.0100″ or even as small as 0.008″ to avoid undesirable spraying of fluid. In some embodiments, the system can have a minimum aspiration pressure of 1 or 2 inHg absolute, to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally similar features and components described herein may be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145.

For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-14l and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The sensors can be used during therapy to provide feedback for the system (e.g., sensors can be used to detect clogs to initiate a clog removal protocol, or to determine the proper therapy mode based on sensor feedback such as jet pulse sequences, aspiration sequences, etc.). The auxiliary ports can therefore be used to connect to the sensors, e.g., by electrical connection, optical connection, mechanical/wire connection, and/or fluid connection. It is also contemplated that the fluid and auxiliary lumens can be configured to carry and deliver other fluids, such as thrombolytics or radio-opaque contrast injections to the target tissue site during treatment.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order.

In various embodiments, the fluid pressure is generated at the pump (in the console or handle). The fluid is accelerated as it exits the ports at the distal end and is directed to the target clot. In this way a wider variety of cost-effective components can be used to form the catheter while still maintaining a highly-effective device for clot removal. Additional details are provided below.

FIG. 1E, Section B-B, illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145e-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment 1F includes the same four auxiliary ports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the exemplary outer wall 140 and inner wall at the top (e.g., 12 o′clock) portion of the device is larger than a distance between the outer wall and inner wall at the bottom (e.g., 6 o′clock) portion of the device.

Section C-C of FIG. 1K illustrates in plan view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 at the base of the funnel that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values.

The manifold is configured to increase a fluid pressure and/or flow rate of the fluid. When fluid is provided by the fluid delivery mechanism to the fluid lumen(s) at a first pressure and/or a first flow rate, the manifold is configured to increase the pressure of the fluid to a second pressure and/or is configured to increase the flow rate of the fluid to a second flow rate. The second pressure and/or second fluid rate can be higher than the first pressure and/or first flow rate. As a result, the manifold can be configured to increase the relatively low operating pressures and/or flow rates generated by the fluid delivery mechanism to the relatively high pressures and/or high flow rates generated by the ports/fluid streams.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which multiply with respect to individual fluid streams that are not directed to combine at the intersection. The increased fluid momentum and/or energy transfer at an intersection may advantageously fragment thrombus more efficiently and/or quickly. As described above, in some embodiments, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15 m/s to up to 150 m/s. At these higher velocities (e.g., above 15 m/s, or alternatively above 20 m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams should be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e., speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

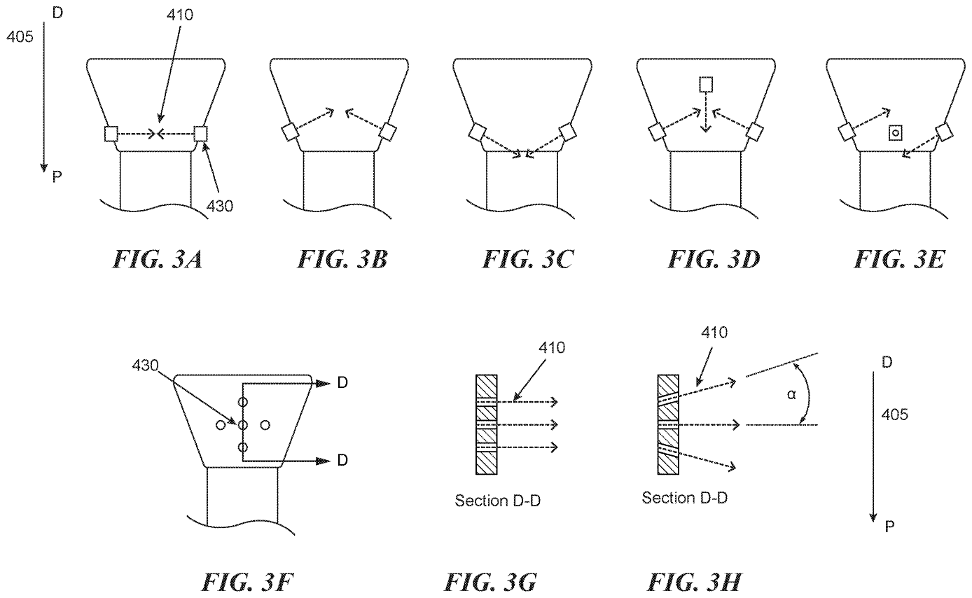

FIGS. 3A-3H depict various configurations of fluid streams 410 that are directed from respective ports 430. A fluid stream 410 can be directed along a path that is substantially orthogonal, proximal, and/or distal to the flow axis 405. In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle a may characterize an angle that a fluid stream 410 is directed with respect to an axis that is orthogonal to the flow axis 405 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

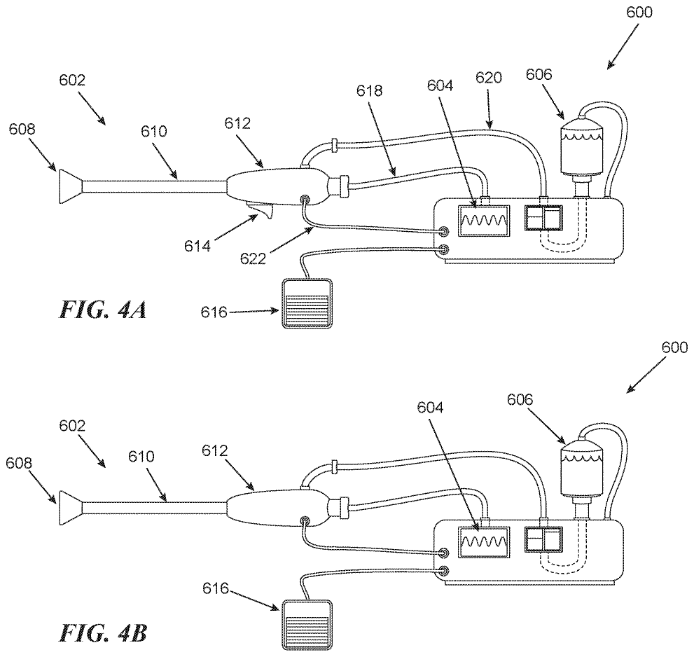

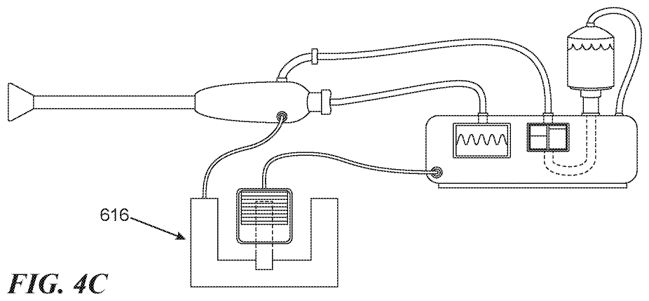

FIGS. 4A-4C illustrate various configurations of a thrombus removal system 600, including a thrombus removal device, 602, a vacuum source and cannister 604, a fluid source 606, and a pump. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 602 can include a funnel 608, a flexible shaft 610, a handle 612, and one or more controls 614 and 616. For example, in the embodiment shown in FIG. 4A, the device can include a finger switch or trigger 614 and a foot pedal or switch 616. These can be used to control aspiration and irrigation, respectively. Alternatively, as shown in the embodiment of FIG. 4B, the device can include only a foot switch 616, which can be used to control both functions, or in FIG. 4C, the device can include only an overpedal 616, also used to control both functions. It is also contemplated that an embodiment could include only a finger switch to control both aspiration and irrigation functions. As shown in FIG. 4A, the vacuum source and cannister 604 can be coupled to the aspiration lumen of the device with a vacuum line 618. Any clots or other debris removed from a patient during therapy can be received by, and stored in, the vacuum cannister 604 for later disposal. Similarly, the fluid source 606 (e.g., a saline bag) can be coupled to the fluid lumens of the device with a fluid line 620 for delivery of high-pressure and velocity fluid streams or jets at the base of funnel 608 to fragment thrombus material engaged by funnel 608, as described above.

Still referring to FIG. 4A, electronics line 622 can couple any electronics/sensors, etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both systems in response to sensor data, such as pressure data, flow data, etc.

Prior to introduction of the thrombus removal device into a patient's blood vessel, the system is primed to remove air by pumping fluid through fluid lumen 45, manifold 25, ports 30, aspiration lumen 55, and vacuum line 618. During a thrombus removal procedure, the thrombus removal device is advanced into the patient's blood vessel, and the funnel is expanded to engage the thrombus. Pressurized fluid is delivered from fluid source 606 and pump through fluid line 620 and fluid lumen 45 to manifold 25 and ports 30. Simultaneously, vacuum is applied to aspiration lumen 55 by vacuum source and cannister 604. When a clot is engaged, the material flowing proximally through aspiration lumen 55 to the vacuum source and cannister is primarily fluid delivered through ports 30 combined with any blood that is able to pass around the engaged thrombus. As the fluid jets begin to break up the thrombus, thrombus material is pulled proximally through aspiration lumen 55 along with the injected fluid and any blood that can pass into the funnel around the thrombus. After the thrombus has been broken up sufficiently to become dislodged from the blood vessel, the remaining thrombus material moves proximally toward the vacuum source and cannister 604.

In some embodiments, the controller can reduce the strength of the vacuum applied to the aspiration lumen after a thrombus has been detected in the vacuum line so that blood loss is minimized.

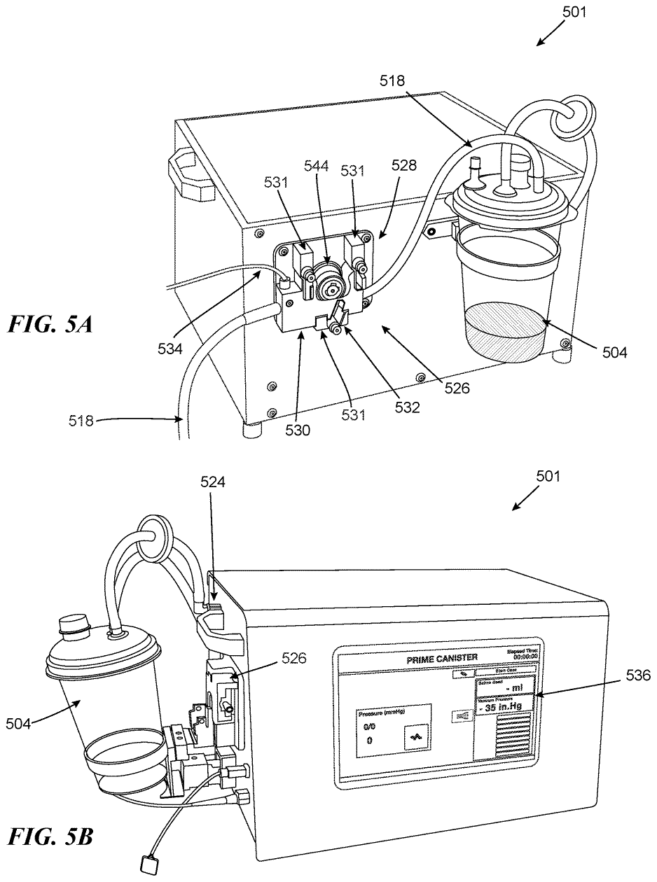

FIGS. 5A-5B show embodiments of a console 501 of a thrombectomy system. The console can be configured to be coupled or attached to a thrombectomy device, such as a thrombectomy catheter as previously described. The console can include an attachment for a cannister 504. A vacuum source (not shown, but disposed in the console) can be controlled by one or more processors of the console 501 to pull a vacuum from an aspiration lumen of the thrombectomy device through or into the cannister 504, resulting in blood and/or clots removed from the thrombectomy device being captured in the cannister. In some embodiments, the cannister 504 is disposable/single-use and can be affixed or mounted to an external mount on the console, and the vacuum source can be re-usable or permanently disposed within the console itself. A vacuum line 518 can couple the vacuum source and cannister 504 to the aspiration lumen of the thrombectomy device or catheter.

As shown in FIGS. 5A-5B, the vacuum line 518 can couple the catheter to the vacuum cannister, and can further couple the cannister to the vacuum source inside the console, such as at vacuum connection 524 in FIG. 5B. The console 501 can also include an integrated display 536 (FIG. 5B), or alternatively, can utilize an external display such as a computer monitor or television that is plugged into the console. The display can guide the user through a thrombectomy procedure and also provide valuable information/feedback to the user, as will be described in more detail below.

The console 501 can further include a pinch valve or electronically controlled clamp assembly 526 that is configured to interface with the vacuum line 518. In some aspects, the electronically controlled clamp assembly 526 is operatively coupled to and controlled by one or more processors of the console. The pinch valve or electronically controlled clamp assembly can be configured to rapidly close or open the vacuum line 518 depending on if aspiration in the thrombectomy catheter is desired or not. This allows the vacuum source to run to create vacuum in the line while also allowing for rapid control over aspiration. In the illustrated embodiment, the pinch valve of clamp assembly 526 can comprise a pump head 544, which can be actuated or controlled to selectively clamp the tubing within the cassette 530.

The console and/or the electronically controlled clamp assembly 526 can include a mounting bracket 528 disposed on the console that is configured to receive a vacuum cassette 530. The vacuum line 518 can pass through or be fluidly coupled to the cassette. When the vacuum cannister 504 is mounted to the console, the vacuum line cassette 530 can be placed within, mounted on, or inserted into the mounting bracket 528 to bring the vacuum line 518 into operable contact with the pinch valve or electronically controlled clamp assembly 526. In some examples, the cassette 530 snaps or is pressure fit into place in the mounting bracket 528. The mounting bracket can optionally include alignment features 531 to enable easy mounting of the cassette to the bracket. For example, one or more guides, tabs, brackets, blocks, posts, or other alignment features can be configured to receive or interface with one or more surfaces of the cassette 530. In some embodiments, the assembly and/or the alignment features can include one or more retention pins or mechanisms 532 to hold the cassette in place within or against the mounting bracket.

Also shown in FIG. 5A, the cassette can include a pressure transducer coupled to a pressure sensor electrical wire 534 extending out of the cassette to be plugged into or electrically coupled with the console. The pressure transducer can be disposed within the cassette 530 and/or within or coupled to the vacuum line 518. The pressure transducer is configured to facilitate pressure measurement within vacuum line 518, and therefore within the aspiration line/lumen of the thrombectomy device.

In some aspects, the pressure transducer can be configured to measure pressure within the vacuum line/aspiration lumen, regardless of if the aspiration is on or off. For example, the pinch valve can be closed or clamped to turn aspiration off and opened or released to turn aspiration on. As described above, the pinch valve can be periodically actuated, or actuated according to a preset interval or time domain. Similarly, the pressure transducer itself can be actuated or controlled to continuously, periodically, or intermittently sense pressure within the lumen(s). In some embodiments, the pressure transducer can be controlled to sense pressure within the aspiration lumen/vacuum line according to preset sensing intervals. In other embodiments, the pressure transducer can measure pressure in a time domain, or at a specified frequency.

In some embodiments, the sensing intervals of aspiration (as controlled by the pinch valve) and of pressure measurement (as controlled by the pressure transducer sensing intervals) can be coordinated. For example, in one embodiment, a sensing interval of the pressure transducer can be timed or controlled to coincide with an aspiration interval (e.g., when the aspiration is turned on by opening or releasing the pinch valve). In other embodiments, a sensing interval of the pressure transducer can be timed or controlled to apply pressure measurements during pulsing of the pinch valve when both aspiration is turned on and when it is turned off. For example, if aspiration is turned on every two seconds (e.g., on for one second, off for one second, and so forth), then the pressure sensor can be pulsed to obtain pressure measurements every one second such that measurements are taken during both the aspiration on and off periods. Any conceivable pulsing sequence for both aspiration and pressure sensing can be implemented. For example, the pressure transducer may be configured to obtain pressure measurements every time aspiration is activated, but only periodically obtain pressure measurements when the aspiration is closed or turned off. Additionally, pressure measurements may be timed to measure pressure only after aspiration has been turned on for a preset period of time (e.g., 1 second after aspiration is activated).

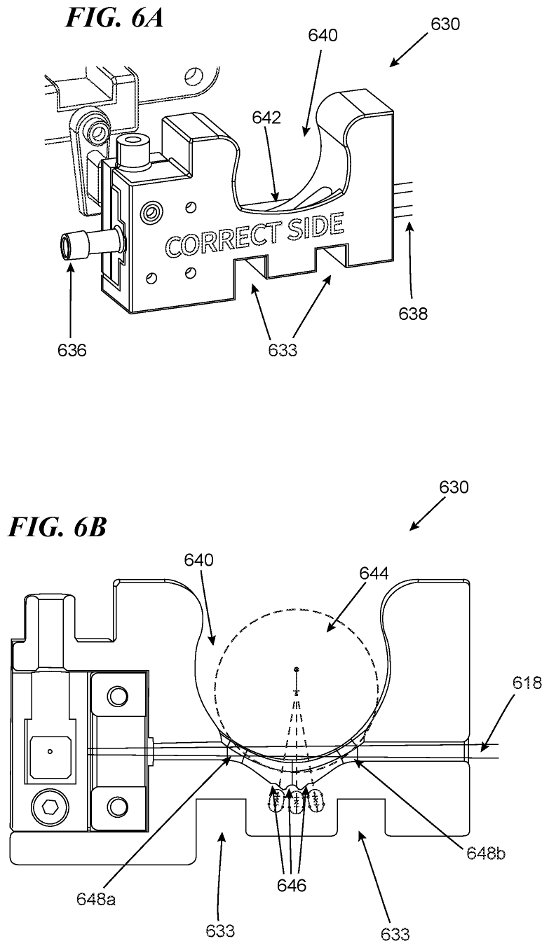

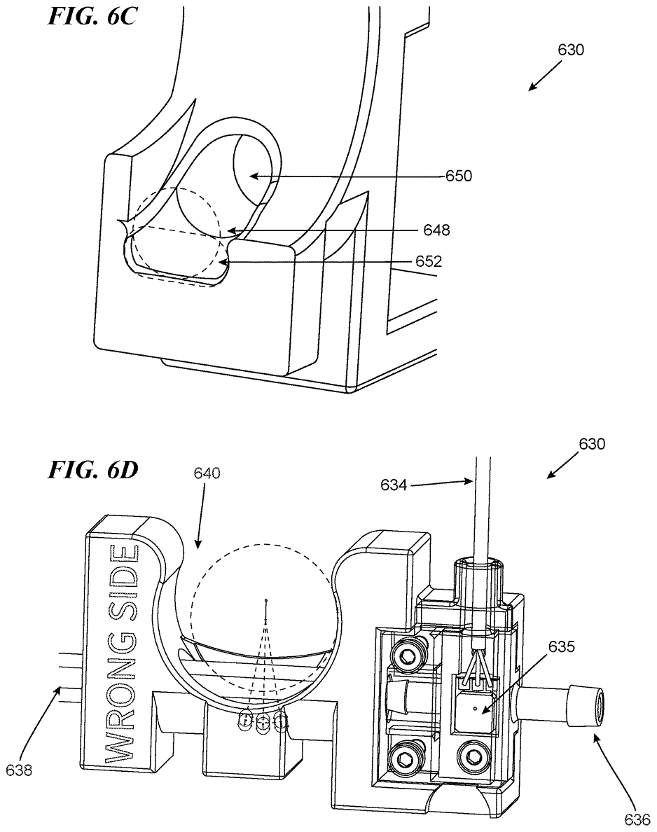

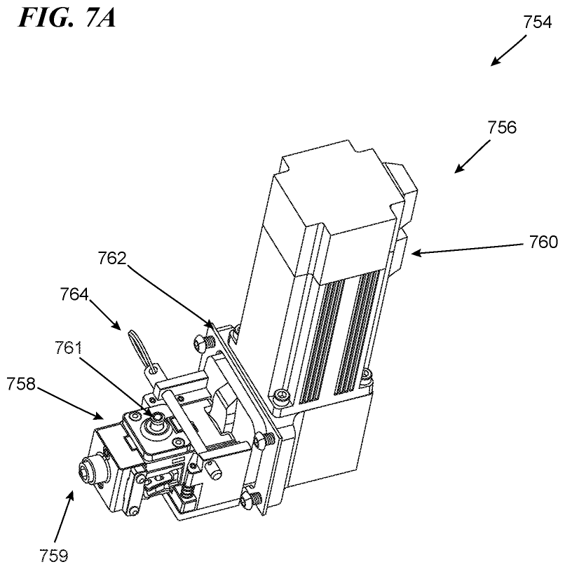

FIGS. 6A-6D illustrate views of a vacuum cassette 630 configured to interface with the console and integrate a pressure sensor/transducer and pinch valve into the system. FIGS. 6A-6B show a view of the vacuum cassette 630 from the side that faces away from the console. For purposes of this disclosure, cassettes labeled with “correct side” indicate the side of the cassette that faces the user when inserted into the mounting bracket of the console. This indicates to the user that the “correct side” is facing the user, and properly orients the cassette against the mounting bracket on the console. The cassette can include an inlet 636 and an outlet 638. The inlet can be coupled to the thrombectomy device with the vacuum line, and the outlet 638 can be coupled to the vacuum line and vacuum source and cannister as previously described. In some embodiments, the cassette itself can include an additional portion of tubing that spans between the inlet and the outlet. This portion of tubing can be referred to as the pinch valve portion of the vacuum line, and can be configured to interface with the pinch valve/pump head during operation of the thrombectomy system.

The cassette 630 can further comprise an opening or cutout 640 configured to interface with a pump head of the electronically controlled clamp assembly (e.g., pump head 544 in FIG. 5A). Inserting the cassette into the mounting bracket on the console aligns the cutout 640 with the pump head, and also places the pump head in operative communication with a pinch valve portion 642 of the vacuum line. Surfaces 633 on the cassette are configured to engage or align with alignment features of the mounting bracket, such as alignment features 531 in FIG. 5A.

FIG. 6B shows the relationship between the pump head 644 and the cutout 640 when the cassette is inserted into the mounting bracket of the console. During loading, the cassette and pinch valve portion of the vacuum line can be inserted into the mounting bracket without compression between the pump head 644 and the vacuum line 618. During operation or clamping of the pump head, the pinch valve portion of the vacuum line 618 can be compressed by a bearing head of the pump against a plurality of protrusions 646 in the cassette. In this example, three protrusions are shown, but any number of protrusions is contemplated, including more or less than three protrusions. The protrusions form features against which the bearing head is able to pinch or clamp off the vacuum line. Additional protrusions beyond three can be redundant, but allow more forgiveness between alignment of the cassette to the mounting bracket and pump head.

In FIG. 6C, an additional optional feature is shown in which the cassette includes a tube path 648 that is lofted to match the shape of the tube. Therefore, the tube path 648 through the cassette can include a location 650 that corresponds to a circular cross sectional shape, for when the vacuum line is not compressed or clamped, and also a location 652 with an ellipsoidal shape, for when the vacuum line is clamped or compressed (against the protrusions), resembling the shape and diameter of the tubing when it is crushed or clamped. The tube path allows for more effective clamping in the clamping region of the cassette, while allowing for the tube to fully open into its circular cross-section when unclamped for better aspiration performance.

In some embodiments, referring to FIG. 6B, the cassette can include a pair of these tube paths on either side of the opening 640 or pump head. For example, pinch valve portion of the vacuum line can span between the inlet and outlet of the cassette, and be configured to pass through tube path 648a on a first side of the pump head and tube path 648b on a second side of the pump head. Both tube paths 648a and 648b can include a portion with a circular cross sectional shape (e.g., location 650) and a portion with a non-circular cross sectional shape (e.g., ellipsoidal location 652). When the pinch valve portion of the tubing is compressed, it can be pressed or compressed into the non-circular cross sectional shape of the cassette. When the pinch valve portion is not compressed, the tube can expand to fill the circular cross sectional shape of the cassette.

FIG. 6D shows a view of the cassette 630 from the “wrong side”, which is the side of the cassette that is placed into contact with the mounting bracket of the console. This includes a cross-sectional view showing the pressure sensor electrical wire 634 electrically coupling the pressure transducer 635 to the console. The pressure transducer/sensor can be any known pressure sensor in the art, including MEMS, resistive, capacitive, piezoelectric, optical, or fluidic pressure sensors. The electrical wire 634 extends out of the cassette and can be plugged into a separate connection on the console. Since the cassette is designed and configured to be disposable or single use, it follows that the integrated pressure sensor and electrical wire are also disposable or single use along with the cassette. As shown in FIG. 6D, the pressure transducer 635 can be positioned between the inlet 636 of the cassette and the opening 640 that is configured to receive the pinch valve/pump head. Therefore, when the pinch valve portion of the tubing is pinched or clamped off by the pinch valve, the pressure transducer is positioned between the pinch valve and the aspiration lumen of the thrombectomy device. This allows the pressure transducer to measure aspiration lumen pressure when the pinch valve is opened or closed.

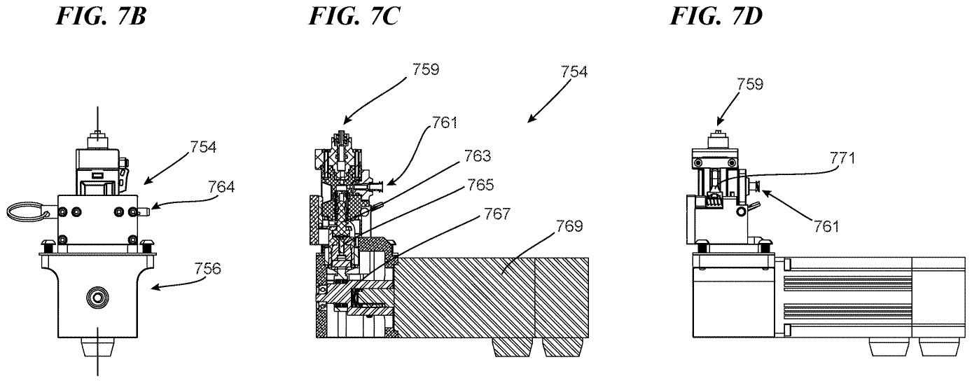

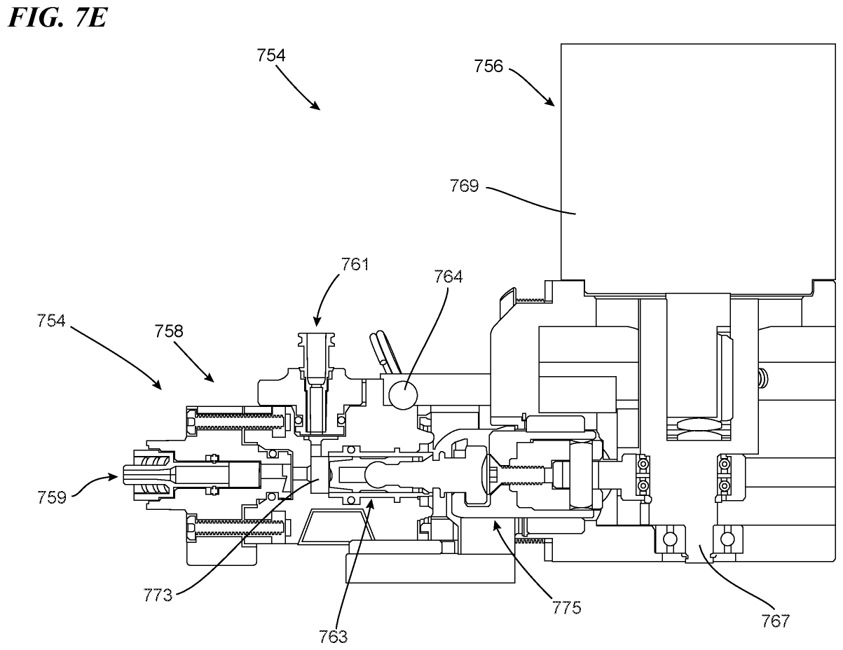

FIGS. 7A-7E illustrate views of an irrigation pump 754 configured to deliver irrigation fluid and/or jetting fluid into a thrombectomy catheter from the console. Inlet 761 can be fluidly coupled to a fluid source, such as a source of saline (not shown). Outlet 759 of the pump 754 can be coupled to an irrigation or fluid line that can be fluidly coupled to aspiration lumens/ports of the thrombectomy device, to provide fluid from the fluid source to the device. Although not shown in the images of the console in FIGS. 5A-5B, it should be understood that the irrigation pump 754 can also include permanent and disposable components. For example, a permanent pump component 756, can be mounted on or within the console, and a disposable pump component 758, can be mounted, inserted into, or connected to the permanent pump component and configured to extend out of the console. In FIG. 7A, the permanent pump component 756 can include a pump motor 760 and a mounting bracket 762. For example, the pump motor 760 can be positioned internally to the console, and the mounting bracket 762 can provide access to the pump motor on an external surface of the console. The disposable pump component can be coupled with the permanent pump component and held in place with, for example, a pin or locking mechanism 764.

FIGS. 7B-7D show additional views of the disposable pump component attached to the permanent pump component. FIG. 7B is a bottom view of the irrigation pump 754, which shows the disposable pump component 754 affixed to the permanent pump component 756 with the retaining pin 764.

FIG. 7C shows a cross sectional view of the irrigation pump 754 showing outlet 759 from the disposable pump component, inlet 761, a pump piston 763, a drive arm 765, a crankshaft 767, and the pump motor 769. In some embodiments, only the motor 769 and crankshaft 767 are positioned in the permanent pump component, and the remaining illustrated components are disposed in the disposable pump component. However it should be understood that any of the components illustrated can be disposed in the disposable and permanent pump components, depending on design and cost requirements.

FIG. 7D shows an optional integrated pressure transducer 771 to provide an indication or measurement of pressure in the fluid or irrigation lines. The pressure transducer 771 can be disposed in the fluid path between the inlet 761 and outlet 759.

FIG. 7E is a detailed view of the irrigation pump 754, including the permanent pump component 756 and the disposable pump component 758. This drawing additionally shows the outlet 759, inlet 761, disposable pump cylinder 773, piston assembly 763, piston drive assembly 775, crankshaft 767, retaining pin 764, and motor 769 of the irrigation pump 754. It should be understood that, in use, the disposable pump component is attached or connected to the permanent pump component on the console, joining or coupling the piston assembly 763 of the disposable pump component to the piston drive assembly 775 of the permanent pump component. The inflow can be attached to a fluid source (such as saline), and the outflow can be fluidly coupled to one or more fluid, irrigation, or jetting lines on the thrombectomy device, as previously described above. Operation of the motor causes the crank shaft to drive the piston drive assembly, which causes the piston assembly 763 and cylinder 773 to pump fluid from the fluid source through the outlet into the thrombectomy device.

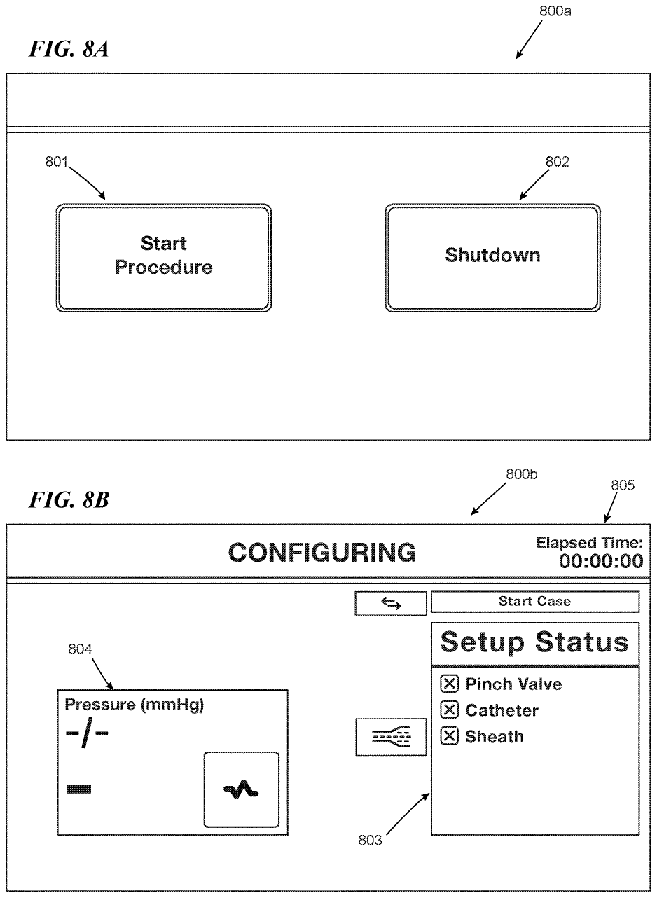

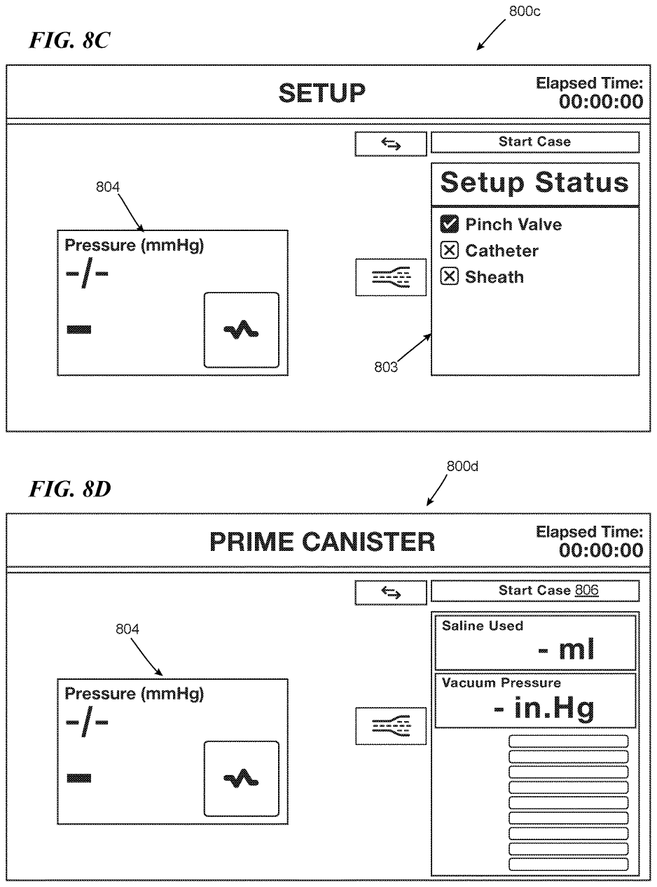

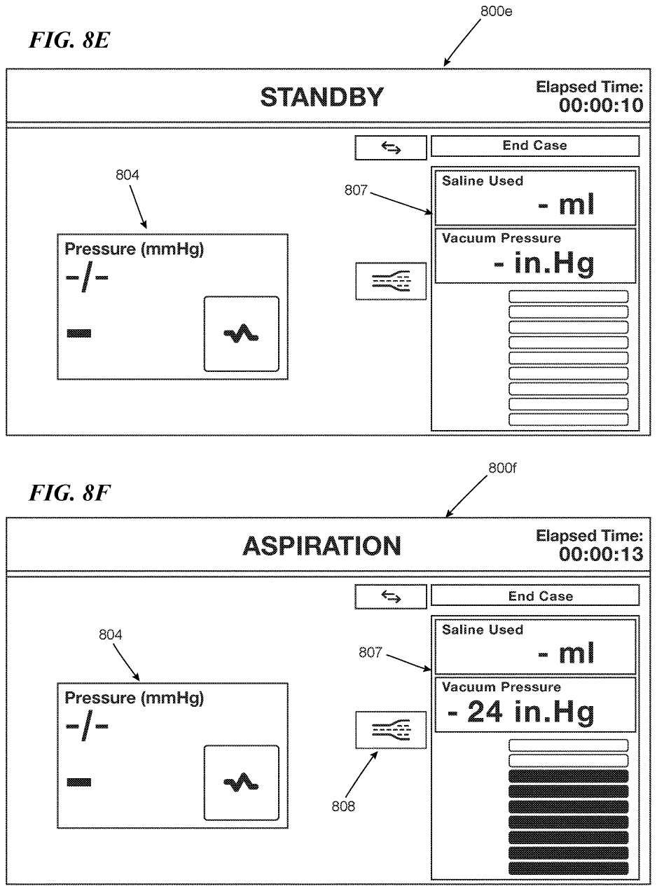

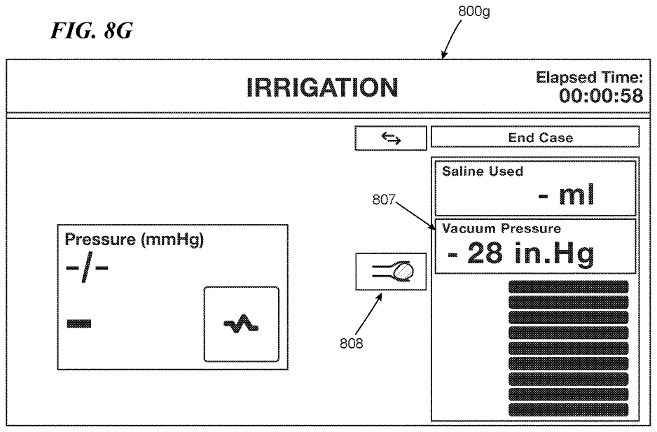

FIGS. 8A-8G illustrate views of a user interface of a thrombectomy console. The views can be displayed on a screen or display of the console, for example. In some embodiments, the display can be a graphical user interface (GUI) that can display information related to the procedure to the patient and also receive input from the user.

In FIG. 8A, an initial screen 800a of the display can display prompts 801 and 802 that give the user the option to start a new procedure (e.g., a thrombectomy procedure) or shutdown the system.

In FIG. 8B, a screen 800b of the display can guide the user through a setup process prior to a thrombectomy procedure. In this example, the screen can display a setup status for a number of system components, particularly components that need to be coupled or hooked up to a system console prior to a procedure. For example, FIG. 8B indicates to a user at prompt 803 that the pinch valve (e.g., vacuum cassette) is not setup, and the catheter and introducer sheath are also not connected to the console. The prompt can indicate if components are not connected with an “X” or other similar symbol, and can provide another indicator or symbol such as a checkmark when the components are connected. This screen also shows that no pressure reading is being measured by the system at prompt 804. At prompt 805, the screen shows the elapsed time of the procedure. In this example, the prompt 805 indicates that he procedure has not yet started (e.g., an elapsed time of 00:00:00).

In FIG. 8C, the screen 800c shows that prompt 803 on the setup status has been updated to show that the pinch valve (e.g., vacuum cassette) has been hooked up properly (as indicated by the check mark) and is recognized by the console/system. The catheter and sheath are still not connected in this example (as indicated by the “X”), and there is still no pressure reading at prompt 804.

FIG. 8D shows a screen 800d after the catheter has been connected, and the system begins to guide the user through a priming process that primes relevant portions of the device (e.g., catheter, cannister, etc.) with an appropriate priming solution such as saline to prevent introduction of air or bubbles into the patient's bloodstream. After the system has been primed, a user can start a case (e.g., initiate thrombectomy) with prompt 806.

FIG. 8E shows a screen 800e of the display after a procedure has started. There is still no pressure measurement at prompt 804, and vacuum and aspiration/jetting have not started yet as indicated by a lack of pressure measurements or saline use in prompt 807.

In FIG. 8F, screen 800f shows that aspiration has been activated in the thrombectomy catheter. The display shows the saline used and vacuum pressure at prompt 807, along with an indicator 808 showing a view of the device (e.g., the funnel), and the current vacuum pressure in the aspiration line at prompt 804 if aspiration line pressure is being measured.

FIG. 8G shows a screen 800g of the display in which a clot has been engaged with the thrombectomy device, such as within the funnel. The indicator 808 showing a view of the device has been updated to show a clot disposed at least partially within the funnel of the device. It can also be seen that the vacuum pressure at prompt 807 has changed as a result of the clot engagement (relative to the vacuum pressure of prompt 807 in FIG. 8F). In some embodiments, the prompt 808 can further display updates the state to “engaged,” “clogged,” “captured,” or the like, indicating approximation of the clot to the funnel prior to the irrigation step. With the clot engaged, the system can be initiated to proceed with jetting/irrigation to assist in breaking up the clot and aspirating the clot from the patient.

While the embodiments herein have been described as being intended to remove thrombi from a patient's vasculature, other applications of this technology are provided. For example, the devices described herein can be used for breaking up and removing hardened stool from the digestive tract of a patient, such as from the intestines or colon of a patient. In one embodiment, the device can be inserted into a colon or intestine of the patient (such as through the anus) and advanced to the site of hardened stool. Next, the aspiration system can be activated to engage the hardened stool with an engagement member (e.g., funnel) of the device. Finally, the jets or irrigation can be activated to break off pieces of the hardened stool and aspirate them into the system. Any of the techniques described above with respect to controlling the system or removing clots can be applied to the removal of hardened stool.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology.

Accordingly, the systems described herein are not limited to those configurations expressly identified, but rather encompasses variations and alterations of the described systems.

### Conclusion

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order.

The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

A complete listing of the claims follows:

1. A thrombus removal system, comprising:
a thrombectomy catheter having an elongate shaft, an aspiration lumen extending along the elongate shaft, and at least one fluid lumen extending along the elongate shaft;
a console configured to be coupled to the thrombectomy catheter, the console including:
a vacuum source adapted to be fluidly coupled to the aspiration lumen of the thrombectomy catheter with a vacuum line;
an irrigation source and pump configured to provide a flow of fluid to the at least one fluid lumen of the thrombectomy catheter with a fluid line; and
a pinch valve assembly configured to receive the vacuum line, wherein the pinch valve assembly is configured to selectively close off the aspiration lumen from the vacuum source.

2. The system of claim 1, wherein the pinch valve assembly comprises a pinch valve disposed on the console.

3. The system of claim 2, wherein the pinch valve assembly is configured receive a vacuum line cassette that includes a pinch valve portion of the vacuum line configured to interface with the pinch valve on the console.

4. The system of claim 3, wherein the vacuum line cassette includes a pressure transducer configured to measure a pressure in the aspiration lumen of the thrombectomy catheter.

5. The system of claim 4, wherein the pressure transducer comprises a MEMS pressure transducer.

6. The system of claim 4, further comprising a pressure transducer wire couped to the pressure transducer and extending out of the vacuum line cassette.

7. The system of claim 6, wherein the console includes a pressure wire connector configured to receive the pressure transducer wire to operatively couple the pressure transducer to the console.

8. The system of claim 3, wherein the vacuum line cassette is disposable.

9. The system of claim 1, wherein the irrigation pump is at least partially disposed in the console.

10. The system of claim 1, wherein the irrigation pump comprises a disposable component and a permanent component.

11. The system of claim 1, further comprising a display configured to guide a user through a thrombectomy procedure.

12. The system of claim 1, wherein the display is configured to present an indicator showing a status of thrombectomy catheter components that are connected to the console.

13. The system of claim 1, wherein the display is configured to present an indicator showing a clot engagement status of the thrombectomy catheter.

14. A thrombectomy system console, comprising:
a vacuum source;
a pinch valve assembly comprising a pinch valve and a mounting bracket, the pinch valve assembly being configured to receive a vacuum cassette having an inlet fluidly coupled to a thrombectomy device and an outlet fluidly coupled to the vacuum source, the pinch valve being operatively coupled to a portion of tubing in the vacuum cassette when the vacuum cassette is inserted into the mounting bracket; and
one or more processors operatively coupled to the pinch valve assembly and configured to selectively couple the vacuum source to the thrombectomy device.

15. The system console of claim 14, wherein the vacuum cassette is fluidly coupled to an aspiration lumen of a thrombectomy device.

16. The system console of claim 14, wherein the pinch valve comprises a pump head.

17. The system console of claim 14, wherein the mounting bracket comprises one or more alignment features configured to interface with one or more corresponding surfaces of the vacuum cassette.

18. The system console of claim 14, wherein the vacuum cassette includes an inlet and an outlet.

19. The system console of claim 18, wherein the inlet is coupled to a vacuum line that extends to an aspiration lumen of the thrombectomy device.

20. The system console of claim 18, wherein the outlet is coupled to a vacuum line that extends to the vacuum source.

21. The system console of claim 16, wherein the pump head fits within an opening of the vacuum cassette.

22. The system console of claim 18, further comprising a pressure transducer disposed in the vacuum cassette between the inlet and the outlet.

23. The system console of claim 22, further comprising a pressure transducer wire extending from the pressure transducer out of the vacuum cassette.

24.-36. (canceled)
